(19) United States
(12) Patent Application Publication
(10) Pub. No.: US 2005/0176679 A1
(43) Pub. Date: Aug. 11, 2005

(54) Process for the preparation of free flowing pulverized adsorbates of tibolone

(76) Inventor: Klaus Glaenzer, Hamburg (DE)

Correspondence Address: J C PATENTS, INC., 4 VENTURE, SUITE 250, IRVINE, CA, 92618, US

(21) Appl. No.: 11/051,095

(22) Filed: Feb. 4, 2005

(30) Foreign Application Priority Data
Feb. 6, 2004 (EP) 04002682.5

(51) Int. Cl. A61K 31/724; A61K 31/716; A61K 31/56

(52) U.S. Cl. 514/57; 514/58; 514/60; 514/171

(57) ABSTRACT

The present invention concerns a process for the preparation of adsorbates and solvates of tibolone where one starts with a solution of tibolone or one of its solvates and/or one of its metabolites in at least one organic solvent where the total water content of the solvent is not higher than 15% by volume, preferably not higher than 5% by volume, disperses therein an adsorbing material selected from the group consisting of celluloses, cellulose derivatives, polyols, sugars, sugar derivatives, maltodextrins, cyclodextrins, starches, polydextroses, or their mixtures, and removes the solvent.

### CROSS-REFERENCE TO RELATED APPLICATION

This application claims benefit of priority of European Patent Application No. 04 002 682.5 filed on Feb. 6, 2004, which is incorporated herein by reference in its entirety for all purposes.

### BACKGROUND OF THE INVENTION

1. Field of the Invention

The present invention relates to a novel process for the preparation of adsorbates of tibolone and its solvates. The invention further relates to tibolone adsorbates and solvates that can be prepared by said process. The invention also relates to pharmaceutical formulations prepared while employing said tibolone adsorbates and solvates. Preferred drug formulations according to the present invention are tablets, capsules, pellets, and granules prepared with the conventional, pharmaceutically acceptable adjuvants in processes known per se. Particularly preferred according to the invention are tablets rapidly releasing the active ingredients that are prepared by direct compression of the tibolone adsorbates and solvates according to the invention.

2. Description of Related Art

The medicinal substance known by the INN tibolone is familiar to chemists under the synonym of 17-hydroxy-7α-methyl-19-nor-17α-pregn-5(10)-en-20-in-3-one (IUPAC) (for the formula see FIG. 1). Tibolone is a synthetic steroid resembling norethisterone and having combined estrogenic, androgenic, and gestogenic activities, more particularly via its active metabolites. The two hydroxy metabolites (3α and 3β) (for the formulas see FIGS. 3 and 4) having activities similar to that of estrogen, while the Δ⁴ isomer (for the formula see FIG. 2) reveals predominantly gestogenic and androgenic activities owing to its binding to gestogen and androgen receptors (cf. www.wechseljahre.com). 7α-Methyl- 17α-ethinylestradiol (for the formula see FIG. 5), a further metabolite identified recently, has a strong estrogenic activity (cf Steroids 67 8 (2002), 681-686). In daily doses of 2.5 mg, tibolone has therapeutic uses, amongst others, for hormone substitution in the postmenopause (on this point see, for instance, Drugs Fut. 6, 302 (1998)).

The manufacture of pharmaceutical preparations with a low-dosage active ingredients as needed in the case of tibolone is a general problem necessitating technical efforts which in part are considerable. The simple solution of a direct compression of powder mixtures implies the risk of separation processes leading to an inhomogeneous distribution of the active ingredients that is pharmaceutically inacceptable. Active ingredients that have been micronized, preground or premilled are for instance used in order to reduce fluctuations in the percentage of active ingredients and keep them within limits admissible according to the pharmacopeia. In other technologies, active ingredients dissolved in organic or aqueous solvents or distributed as microparticles in these solvents are processed by wet granulation, or are sprayed onto carriers in prior process steps. Granules that can be pressed are finally obtained after drying, comminuting, and classifying.

In EP 0 657 161 B1, for example, the above problem of fluctuations in the content of the active ingredients in tablets has been described, viz, active ingredients and steroids in particular migrating into the packaging material, which often consists of PVC material, thus causing a drop in the content of active ingredients during storage of the tablets. In EP 0 657 161 B1, this problem is solved by dispersing or dissolving both the steroid—desogestrel in combination with ethinylestradiol—and a lubricant such as talc, magnesium stearate, or stearic acid in an organic solvent, then mixing this suspension or solution with a carrier comprising lactose, polyvinylpyrrolidone and a disintegrant so as to produce a mixture of steroid solution and carrier material, thereafter removing the organic solvent so as to produce granules with a surface film inhibiting migration of the steroid combination contained in the granules. For desogestrel in combination with ethinylestradiol, very good migration values, that is, almost no migration of the steroid, have been achieved according to EP 0 657 161 B1 for coated tablets after a time of storage of 19 months at 32° C. and 70% relative humidity.

Still in the above context, though, different technologies are described in EP 0 929 294 B1, that is, spraying of the active ingredients as a solution or dispersion in prior process steps onto a free-flowing and/or directly pressable powder system on carrier materials. The sprayed powder system is then processed to tablets, capsules, or sachets. The active ingredients reported in EP 0 929 294 B1 are ethinylestradiol and desogestrel.

Those skilled in the art know the corresponding procedures, which are technically demanding and work-intensive (see, for instance, in Die Tablette [The Tablet], W. A. Ritschel and A. Bauer-Brandl, 2^{nd} ed., ECV-Editio Cantor publishers, 2002) and will be able to tackle the problem of an inhomogeneous distribution of low-dosage active ingredients in individual cases. Apart from the large technical effort, however, these known processes are associated with important additional disadvantages and special problems in the case of tibolone.

These are caused more particularly by the instability of tibolone as a substance. Under the effects of heat, acidic or oxidative interactions that cannot be avoided during these complex production steps, the corresponding degradation processes already start while manufacturing the drug formulation. This is particularly pronounced in all granulation procedures or when spraying solutions of tibolone. In this way decomposition and/or isomerization processes are induced which give rise to insufficient stability of the final product. With the intention of obtaining more stable formulations, stabilizing additives with pH-controlling and antioxidant properties have been proposed according to WO 03/032924, and described in the instance of pregrinding with sodium bicarbonate or sodium citrate followed by wet granulation.

Apart from the transformation to stereoisomeric compounds in the dissolved state that have already been mentioned, the morphology obtained upon precipitation or crystallization is another special feature of tibolone. Depending on the conditions of drying, precipitation, or crystallization, different solids or morphologies are obtained. Depending on the solvent systems, for instance, polymorph I, polymorph II, amorphous material, or their mixtures can be obtained. The process of crystallization and the phase transitions depend on a large number of factors that are difficult to control (see, for instance, S. X. M. Boerrigter et al, J. Phys. Chem. B, 4725 -4731 (2002); 14^{th} Int. Symp. on Industrial Crystallization, Cambridge (1999); EP-A-389 035). It must be noted in particular that minor changes in the conditions of crystallization, in the solvent systems, in the degree of saturation, in the seeding crystals, and conditions of drying will strongly influence the formation of a particular morphology. Apart from this uncertain, poorly defined initial mixing ratio of polymorphic formulations, phase changes that can occur under particular conditions during subsequent storage constitute a further quality problem that is difficult to control. It must be remembered in this context that different polymorphs have a different dissolution behavior. This in turn will produce different, variable dissolution profiles of the drugs manufactured from them (see EP-A-389 035). It can be concluded, therefore, that there is a very high risk of uncontrolled transformation to polymorphous or isomeric formulations, particularly in processes for the manufacture of pharmaceutical preparations in which the tibolone active ingredient must be pre-dissolved or partly dissolved, so that it is not guaranteed that regulatory requirements as to a clearly defined, active ingredient or constant quality of the drug can be met.

Therefore, known processes for the preparation of pharmaceutical formulations of tibolone require a very large technical effort and are time and cost intensive, provided they are practical at all, and so far they do not solve the problem of a stable, qualitatively acceptable drug formulation, or do not solve it satisfactorily.

### BRIEF SUMMARY OF THE INVENTION

Thus it is an object of the present invention, to develop a simple and economical process for the preparation of defined and stable tibolone powder systems that can be used immediately for manufacturing pharmaceutical formulations, a process that is not restricted, however, to a particularly preferred morphology of the active ingredients and avoids the disadvantages discussed above.

### BRIEF DESCRIPTION OF THE DRAWINGS

FIG. 1 shows the formula of tibolone (17-hydroxy-7α-methyl-19-nor-17α-pregn-5(10)-en-20-in-3-one).

FIG. 2 shows the formula of Δ⁴ isomer of tibolone.

FIG. 3 shows the formula of 3α-hydroxy metabolite of tibolone.

FIG. 4 shows the formula of 3β-hydroxy metabolite of tibolone.

FIG. 5 shows the formula of a further metabolite of tibolone, namely 7α-methyl-17 α-ethinylestradiol.

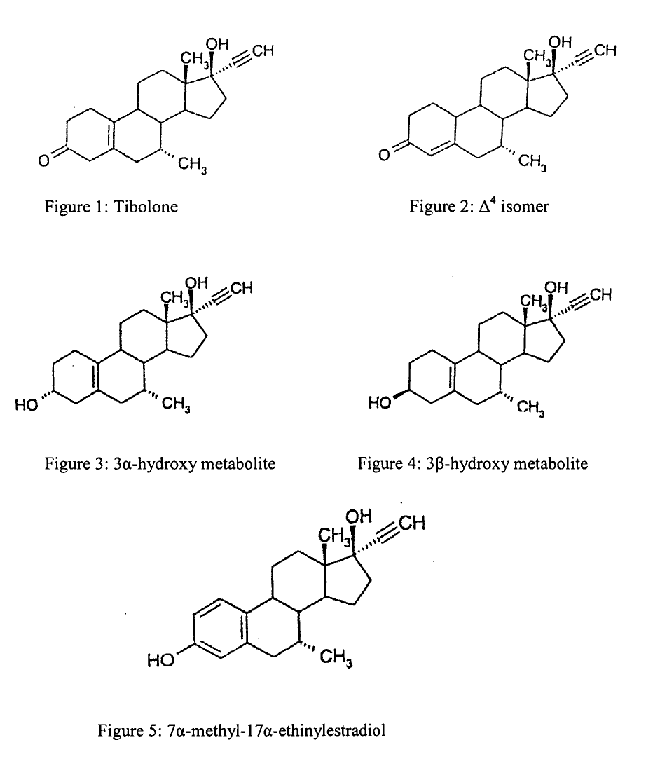

### DETAILED DESCRIPTION OF THE INVENTION

Therefore, the present invention relates to a process for the preparation of adsorbates of tibolone and its solvates according to which process one starts from a solution of tibolone or one of its solvates and/or one of its metabolites in a predominantly organic solvent, disperses the adsorbing material in it, and removes the solvent, which can more particularly be achieved by drying.

The present invention also relates to adsorbates of tibolone and its solvates which can be prepared by the process mentioned above.

According to the invention, the total water content of the solvent is not higher than 15% by volume, preferably not higher than 5% by volume.

The invention further concerns pharmaceutical formulations containing these new tibolone adsorbates and solvates thereof. Where applicable, the pharmaceutical formulations contain further adjuvants and can be converted to the desired drug delivery formulation. Tablets produced by direct compression which rapidly release the active ingredients, and which may be coated, are particularly preferred.

For the process of preparing tibolone adsorbates and solvates thereof according to the invention organic solvents are suitable for the solution containing the active pharmaceutical agent. The organic solvents are more particularly selected from the group of lower alkanols with one to four carbon atoms, group of ethers, esters, group of polyvalent alcohols such as glycol, and group of aliphatic ketones, as well as mixtures of said solvents. Methanol, ethanol, isopropanol, n-propanol, acetone and other solvents such as ethyl acetate, methyl ethyl ketone, MTBE (methyl tert-butyl ether), dichloromethane, petroleum ether, hexane, an acetone/water mixture, an acetone/hexane mixture, an ethyl acetate/hexane mixture, a dichloromethane/ethyl acetate mixture, as well as further mixtures of said solvents are particularly preferred. According to the invention, it is preferred to work under inert gas, for instance under nitrogen, and where necessary using degassed or deoxygenated solutions. Basic substances such as volatile nitrogen bases (for instance alkylamines or even pyridine) can be present as additives in the solutions.

According to the invention, those pharmaceutically acceptable adjuvants are used as adsorbing materials which are appropriate for a rapid release of the active ingredients, such as celluloses and cellulose derivatives, polyols, sugars, sugar derivatives, and more particularly lactose, maltodextrin, starches, cyclodextrins, polydextroses, or mixtures of said substances. Microcrystalline cellulose, lactose, and mannitol are preferred according to the invention. For an improvement of the flow properties, additives containing silica or titaniumdioxid can be used.

According to the invention, the ratio of active pharmaceutical agent to adsorbing material is in the range from 1:0.1 to 1:100, a range from 1:2 to 1:10 being particularly preferred.

All common pharmaceutical adjuvants can be used to prepare the pharmaceutical formulations, for which tablets are more particularly preferred. As fillers and/or binders, for example, celluloses and/or cellulose derivatives (for instance microcrystalline cellulose, native cellulose, hydroxypropyl cellulose, hydroxypropyl methyl cellulose, methyl cellulose), sugars (for instance lactose, fructose, saccharose, glucose, maltose), sugar alcohols (for instance lactitol, mannitol, sorbitol, xylitol), inorganic fillers (for instance calcium phosphates and calcium sulfates), and starches (for instance corn starch, potato starch, wheat starch, dextrins, pregelatinized starches) can be used. Beyond that, all other adjuvants known to those skilled in the art from their basic galenic knowledge, such as lubricants, disintegrants, wetting agents, agents to improve the flow behavior, alkaline additives, stabilizers, as well as flavors, pigments, and dyes, can be used to prepare the drug formulations according to the invention.

The portion of binders in the complete mixture of the drug preparation is preferably between 0 and 20% (mass/mass), the fraction of fillers and adjuvants in the complete mixture is 20 to 99% by weight, preferably 50 to 99% by weight.

The process according to the invention on one hand yields stable adsorbates of tibolone characterized, moreover, by a rapid release of the active ingredients in water under physiological conditions. These tibolone adsorbates are used as carriers of the active pharmaceutical agent in the preparations according to the invention, resulting in a highly constant uniformity of the tibolone content meeting all pharmacopoeial requirements without any problem.

According to the invention, a process has been found which, starting from a solution of tibolone and from its solvates and/or one of its metabolites in an organic solvent, leads to adsorbates of the active ingredients that are suitable for immediate further processing.

In an embodiment of the invention, the solution of tibolone active ingredients can on principle be prepared by dissolving the tibolone and/or its metabolites in a suitable organic solvent; though it is more advantageous to directly use a solution of the active ingredients resulting anyhow during synthesis, without isolation of the tibolone.

Tibolone can for instance be prepared according to Wieland and Anner, Helv. Chim. Acta, 1453-1461 (1967), but omitting the recrystallization steps involving a dissolution in dichloromethane/ether, and instead dispersing the adsorbing material in the solution of the active ingredients, and later removing the solvent. The kind of organic solvent used then results, in any given case, from the final step of synthesis in the process chosen for preparing the active ingredients.

The pharmaceutically acceptable adsorbing material that is then added to the organic solution of the active ingredients, and that is insoluble or poorly soluble in it, is well wetted, and immediately thereafter the solvent is removed. The drying process can be promoted by temperature and by applying a vacuum. Advantageously, it is conducted in such a way that appropriate mechanical action (e.g., rotating, tumbling, or stirring motion) yields a uniform distribution. The solvent can be recovered by working in a closed system, and reused for a subsequent process. According to the invention, a precipitation and isolation of the tibolone is omitted. Adsorbates containing tibolone that have been prepared by the process described can be employed directly in further processing to drug formulations such as tablets, capsules, pellets, or granules, preferably in further processing by a direct compression process.

Optionally, the adsorbates or drug formulations thus obtained can be further provided with coatings of pharmaceutical polymethacrylates such as Eudragit® films, methyl cellulose, ethyl celluloses, hydroxypropyl methyl celluloses, cellulose acetate phthalates and/or shellac in order to meet a specific application, e.g., controlled release of the active ingredients and/or taste masking. Those ordinary skilled in the art have sufficient technical possibilities to accomplish this.

It has been found surprisingly that adsorbates prepared by the process according to the invention contain the active ingredients in the homogeneous distribution required for drugs, and release it without limitations.

In the known crystal structures of the two tibolone modifications (monoclinic and triclinic), the molecules are aggregated to chains via hydrogen bonds. The assumption is plausible that the tibolone chains take up a similar arrangement on the surface of the adsorbates used, and that this arrangement has a stabilizing effect.

The characteristics mentioned are retained as well, more particularly, when the tibolone adsorbates and solvates are processed to drug formulations, e.g., tablets. Moreover, this direct processing does not entail any change in the content of by-products or decomposition products (=sum of all contaminants) in the route from active ingredients to drug formulation (tablet).

The invention will now be explained more closely by the following examples, without however limiting the invention thereto.

### EXAMPLES

Examples 1 to 4

HPLC method for determining the content of active ingredients or sum of all contaminants All HPLC measurements were performed with an Agilent 100 HPLC.

| | |
|---|---|
| Column: | CC 250/4 Nucleosil 100-5 C18 |
| Mobile phase: | 50% aqueous ammonium bicarbonate |
| | solution (0.005%) 50% acetonitrile |
| Flow rate: | 1.0 ml min⁻¹ |
| Detector: | UV, 210 nm |
| Injection volume: | 20 μl |
| Tibolone retention time: | about 13 min |
| Analysis time: | 60 min |

### Example 1: Tibolone-microcrystalline cellulose adsorbate

To a solution of tibolone (0.04 g/ml) in acetone with 0.1% pyridine under nitrogen, microcrystalline cellulose is added in an amount such that the mixture contains 0.12 g microcrystalline cellulose (Celphere SCP-100®) per ml. The solvent then is dried off with continuous agitation in a vacuum (rotating vaporizer or tumble drier) at 25° C. In the end the mixture is post-dried for a short time at 35° C. to remove residual solvent.

Theoretical content of active ingredients in the adsorbate: 25%

| Active ingredients | | |
|---|---|---|
| content by HPLC | Adsorbate (% tibolone) | 100-mg tablet (mg tibolone) |
| Sample No. 1 | 25.0 | 2.53 |
| Sample No. 2 | 24.9 | 2.49 |
| Sample No. 3 | 25.0 | 2.48 |
| Sample No. 4 | 25.1 | 2.50 |
| Sample No. 5 | 24.9 | 2.46 |

From the adsorbate, tibolone tablets were made by direct compression in the following composition:

| | |
|---|---|
| Tibolone-Microcrystalline cellulose adsorbate | 10 mg |
| corresponding to 2.5 mg tibolone | |
| Microcrystalline cellulose (Celphere SCP-100 ®) | 75 mg |
| Adjuvants (croscarmellose sodium, sodium lauryl sulfate, | 15 mg |
| silica, magnesium stearate) in the usual amounts | |

The amounts of further adjuvants used are known to those skilled in the art from their basic knowledge, and can be taken from standard references for tablet formulation, for instance from Ritschel et al., Die Tablette [The Tablet], Editio Cantor, Aulendorf, 2^{nd} ed., 2002.

Properties of the mixture that is ready to be pressed, and of the tablets:

| | |
|---|---|
| Compressibility and flowability: | good |
| Mean hardness: | 86 N |
| Abrasion: | <0.1% (determined according to |
| | Ph. Eur.) |
| Disintegration time: | 60 s (determined according to Ph. Eur.) |
| Release: | 100% after 15 min (determined |
| | according to Ph. Eur., 1000 ml water, |
| | 37° C., paddle at 75 rpm) |
| Content: | see Table |
| Contents uniformity: | meets Ph. Eur. |
| Sum of all impurities | Granules: 0.16%; tablet: 0.18% |
| (referred to the tibolone peak): | |

The tablets thus obtained can be provided with a coating where applicable.

### Example 2: Tibolone-mannitol adsorbate

To a solution of tibolone (0.02 g/ml) in ethanol containing 0.1% triethylamine under nitrogen, mannitol is added in an amount such that the mixture contains 0.18 g mannitol (Mannogem®) per ml. The solvent then is dried off with continuous agitation in a vacuum (rotating vaporizer or tumble drier) at 25° C. In the end the mixture is post-dried for a short time at 35° C. to remove residual solvent.

Theoretical content of active ingredients in the adsorbate: 10%.

| Active ingredients | | |
|---|---|---|
| content by HPLC | Adsorbate (% tibolone) | 100-mg tablet (mg tibolone) |
| Sample No. 1 | 9.95 | 2.51 |
| Sample No. 2 | 9.92 | 2.54 |
| Sample No. 3 | 10.23 | 2.49 |
| Sample No. 4 | 10.11 | 2.51 |
| Sample No. 5 | 10.04 | 2.52 |

From the adsorbate, tibolone tablets were made by direct compression in the following composition:

| | |
|---|---|
| Tibolone-mannitol adsorbate corresponding to 2.5 mg tibolone | 25 mg |
| Mannitol | 60 mg |
| Adjuvants (as in example No. 1) | 15 mg |

Properties of the mixture that is ready to be pressed, and of the tablets:

| | |
|---|---|
| Compressibility and flowability: | satisfactory to good |
| Mean hardness: | 93 N |
| Abrasion: | 0.2% (determined according to |
| | Ph. Eur.) |
| Disintegration time | 82 s (determined according to Ph. Eur.) |
| Release: | 100% after 15 min (Ph. Eur., 1000 ml |
| | water, 37° C., paddle at 75 rpm) |
| Content: | see Table |
| Contents uniformity: | meets Ph. Eur. |
| Sum of all impurities | Granules: 0.21%; tablet: 0.17% |
| (referred to the tibolone peak): | |

The tablets thus obtained can be provided with a coating where applicable.

### Example 3: Tibolone-lactose adsorbate

To a solution of tibolone (0.03 g/ml) in isopropanol containing 0.2% pyridine under nitrogen, lactose (Lactopress® anhydrous) is added in an amount such that the mixture contains 0.21 g lactose per ml. The solvent then is dried off with continuous agitation in a vacuum (rotating vaporizer or tumble drier) at 25° C. In the end the mixture is post-dried for a short time at 35° C. to remove residual solvent.

Theoretical content of active ingredients in the adsorbate: 12.5 %

| Active ingredients | | |
|---|---|---|
| content by HPLC | Adsorbate (% tibolone) | 100-mg tablet (mg tibolone) |
| Sample No. 1 | 12.52 | 2.48 |
| Sample No. 2 | 12.50 | 2.46 |
| Sample No. 3 | 12.53 | 2.49 |
| Sample No. 4 | 12.48 | 2.53 |
| Sample No. 5 | 12.48 | 2.51 |

From the adsorbate, tibolone tablets were made by direct compression in the following composition:

| | |
|---|---|
| Tibolone-lactose adsorbate corresponding to 2.5 mg tibolone | 20 mg |
| Lactose | 65 mg |
| Adjuvants (as in example No. 1) | 15 mg |

Properties of the mixture that is ready to be pressed, and of the tablets:

| | |
|---|---|
| Compressibility and flowability: | satisfactory to good |
| Mean hardness: | 74 N |
| Abrasion: | 0.2% (determined according to |
| | Ph. Eur.) |
| Disintegration time | 80 s (determined according to Ph. Eur.) |
| Release: | 100% after 15 min (Ph. Eur., 1000 ml |
| | water, 37° C., paddle at 75 rpm) |
| Content: | see Table |
| Contents uniformity: | meets Ph. Eur. |
| Sum of all impurities | Granules: 0.16%; tablet: 0.14% |
| (referred to the tibolone peak): | |

The tablets thus obtained can also be provided with a coating.

### Example 4: Tibolone-microcrystalline cellulose adsorbate

To a solution of tibolone (0.05 g/ml) in ethanol with 0.2% pyridine under nitrogen, microcrystalline cellulose is added in an amount such that the mixture contains 0.15 g microcrystalline cellulose (Celphere SCP-100®) per ml. The solvent then is dried off with continuous agitation in a vacuum (rotating vaporizer or tumble drier) at 25° C. In the end the mixture is post-dried for a short time at 35° C. to remove residual solvent.

Theoretical content of active ingredients in the adsorbate: 25%

| Active ingredients content by | Adsorbate | |
|---|---|---|
| HPLC | (% tibolone) | 100-mg tablet (mg tibolone) |
| Sample No. 1 | 25.3 | 2.49 |
| Sample No. 2 | 25.0 | 2.52 |
| Sample No. 3 | 25.0 | 2.55 |
| Sample No. 4 | 24.9 | 2.52 |
| Sample No. 5 | 25.1 | 2.53 |

From the adsorbate, tibolone tablets were made by direct compression in the following composition:

| | |
|---|---|
| Tibolone-microcrystalline cellulose adsorbate | 10 mg |
| corresponding to 2.5 mg tibolone | |
| Microcrystalline cellulose | 75 mg |
| Adjuvants (as in Example No. 1) | 15 mg |

Properties of the mixture that is ready to be pressed, and of the tablets:

| | |
|---|---|
| Compressibility and flowability: | satisfactory to good |
| Mean hardness: | 82 N |
| Abrasion: | 0.12% (determined according to |
| | Ph. Eur.) |
| Disintegration time: | 62 s (determined according to Ph. Eur.) |
| Release: | 100% after 15 min (Ph. Eur., 1000 ml |
| | water, 37° C., |
| | paddle at 75 rpm |
| Content: | see Table |
| Contents uniformity: | meets Ph. Eur. |
| Sum of all impurities | Granules: 0.17%; tablet: 0.16% |
| (referred to the tibolone peak): | |

The tablets thus obtained can also be provided with a coating.

1. A process for the preparation of adsorbates of tibolone and of its solvates, characterized in that one starts from a solution of tibolone or one of its solvates and/or one of its metabolites in at least one organic solvent with a total water content of the solvent of no more than 15% by volume, preferably of no more than 5% by volume, disperses in it an adsorbing material selected from the group consisting of celluloses, cellulose derivatives, polyols, sugars, sugar derivatives, maltodextrins, cyclodextrins, starches, polydextroses, or their mixtures, and removes the solvent.

2. The process according to claim 1, characterized in that the adsorbing material is selected from the group of microcrystalline cellulose, lactose, and mannitol.

3. The process according to claim 1, characterized in that a ratio of active ingredients to adsorbing material is set in the range from 1:0.1 to 1:100, more particularly in the range from 1:2 to 1:10.

4. The process according to claim 1, characterized in that organic solvents with a total water fraction of no more than 15% by volume are used alone or in mixtures as the solvent, the organic solvents being selected from the group of lower alkanols with one to four carbon atoms, the group of ethers, the esters, and the group of aliphatic ketones and their mixtures.

5. The process according to claim 4, characterized in that a solvent from the group consisting of methanol, ethanol, isopropanol, n-propanol, acetone, ethyl acetate, methyl ethyl ketone, methyl tert-butyl ether, dichloromethane, petroleum ether, hexane, an acetone-water mixture, an ethyl acetate-hexane mixture, a mixture of dichloromethane and ethyl acetate is employed as a solvent, the solvent being more particularly kept under inert gas and possibly containing basic additives.

6. The process according to claim 1, characterized in that a solution of the active ingredients is employed which is obtained in the course of tibolone synthesis, and/or a solution of the active ingredients is employed which contains the active ingredients from tibolone metabolism, and the adsorbing material is dispersed in the solution.

7. Adsorbates of tibolone and its solvates, characterized in that they obtainable according to the process according to claim 1.

8. Pharmaceutical formulation with at least one active ingredients and where applicable further, pharmaceutically acceptable adjuvants, characterized in that it contains tibolone adsorbates according to claim 1 as the active ingredients.

9. Pharmaceutical formulation according to claim 8, characterized in that it exists in the formulation of tablets, capsules, pellets, and granules that can be prepared in known manner with common, pharmaceutically acceptable adjuvants.

10. Pharmaceutical formulation according to claim 8 in the formulation of tablets made by direct compression and rapidly releasing the active ingredients.

11. Pharmaceutical formulation according to claim 8, characterized in that the fraction of binder in the total mixture used to prepare the drug is up to 20% (mass/mass).

12. Pharmaceutical preparation according to claim 8, characterized in that the fraction of fillers and adjuvants in the total mixture used to prepare the drug is between 20 and 99% by weight, preferably between 50 and 99% by weight.

13. The process according to claim 2, characterized in that a solution of the active ingredients is employed which is obtained in the course of tibolone synthesis, and/or a solution of the active ingredients is employed which contains the active ingredients from tibolone metabolism, and the adsorbing material is dispersed in the solution.

14. The process according to claim 3, characterized in that a solution of the active ingredients is employed which is obtained in the course of tibolone synthesis, and/or a solution of the active ingredients is employed which contains the active ingredients from tibolone metabolism, and the adsorbing material is dispersed in the solution.

15. The process according to claim 4, characterized in that a solution of the active ingredients is employed which is obtained in the course of tibolone synthesis, and/or a solution of the active ingredients is employed which contains the active ingredients from tibolone metabolism, and the adsorbing material is dispersed in the solution.

16. The process according to claim 5, characterized in that a solution of the active ingredients is employed which is obtained in the course of tibolone synthesis, and/or a solution of the active ingredients is employed which contains the active ingredients from tibolone metabolism, and the adsorbing material is dispersed in the solution.
